(19) United States
(12) Patent Application Publication
(10) Pub. No.: US 2017/0304096 A1
(43) Pub. Date: Oct. 26, 2017

(54) STENT DELIVERY SYSTEM AND METHOD OF MANUFACTURING THE SAME

(71) Applicant: TERUMO KABUSHIKI KAISHA, Tokyo (JP)

(72) Inventor: Teruo AOKI, Fujinomiya-city (JP)

(73) Assignee: TERUMO KABUSHIKI KAISHA, Tokyo (JP)

(21) Appl. No.: 15/648,976

(22) Filed: Jul. 13, 2017

Related U.S. Application Data
(63) Continuation of application No. PCT/JP2015/084005, filed on Dec. 3, 2015.

(30) Foreign Application Priority Data
Jan. 15, 2015 (JP) 2015-005668

(51) Int. Cl. A61F 2/958; A61M 25/10; A61F 2/90; A61F 2/89

(52) U.S. Cl. CPC A61F 2/958; A61F 2/90; A61M 25/1002; A61F 2/89; A61F 2002/9583; A61M 2025/1004; A61M 2025/1056; A61F 2210/0004

(57) ABSTRACT

A stent delivery system includes an elongated shaft, a balloon and a stent. The balloon is dilatable from a contracted position to an expanded position when a fluid flows into an interior of the balloon. The stent is formed by wires that are spaced apart so that at least one gap is formed between the wires. The stent is mounted on the outer surface of the balloon when the balloon is contracted. The balloon includes a protruding portion which protrudes radially outward through the gap of the stent when the stent is mounted on the balloon and the balloon is in the contracted position. A lubricant is disposed on the outer surface of the balloon at least on a part of the protruding portion of the balloon, and an other portion of the balloon covered by the stent does not have the lubricant.

### CROSS-REFERENCES TO RELATED APPLICATIONS

This application is a continuation of International Application No. PCT/JP2015/084005 filed on Dec. 3, 2015, and claims priority to Japanese Patent Application No. 2015-005668 filed Jan. 15, 2015, the entire content of both of which is incorporated herein by reference.

### TECHNICAL FIELD

The present invention generally relates to a stent delivery system used for performing treatment, for example, of a stenosed site or an occluded site inside a body lumen such as a blood vessel. The present invention also generally relates to a method of manufacturing the stent delivery system and a method of use.

### BACKGROUND DISCUSSION

Recently, a method of indwelling a stent in a lesion area (stenosed site) of the coronary artery has been performed, for example, in treatment of acute myocardial infarction and angina pectoris. A similar method is also sometimes performed to improve stenosed sites formed inside other blood vessels, the bile duct, the trachea, the esophagus, the urethra, and other body lumens. A stent delivery system that is used for indwelling a stent generally includes an elongated shaft and a balloon provided on a distal side of the shaft portion. The balloon is radially dilatable (i.e., configured to expand radially outwardly). A tubular stent formed with metal or resin wires is installed (mounted) on an outer circumferential surface of the contracted balloon (i.e., the outer surface of the balloon in the contracted/deflated position). When the balloon is caused to dilate after arriving at a target site inside a body via a thin blood vessel, the stent mounted on the balloon dilates (expands radially outwardly) while being plastically deformed, and a stenosed site is thereby widened. When the balloon is thereafter caused to contract, the stent remains in an expanded state, and the stenosed site is maintained in the widened state (i.e., the inner diameter of the stenosed site is widened by the expanded stent).

Since a stent mounted on the balloon in the stent delivery system has high sliding friction and is unlikely to be bent, passing properties in a case of passing through particularly a bent portion or a stenosed site of a blood vessel are required. For example. Japanese Patent No. 4,663,945 discloses a method of coating a stent protective sleeve with a lubricant for improving sliding characteristics inside a body lumen. The coating is performed in order to improve the passing properties of the stent delivery system.

### SUMMARY

If a part of the outer surface of a balloon (i.e., a balloon covered with a stent) is coated with the lubricant, the stent is more likely to deviate from the balloon when delivering the stent disposed on the outer circumferential surface of the balloon. Therefore, there is a possibility that it becomes difficult to accurately indwell the stent. There is also a possibility that the stent comes off from the balloon when the stent is carried to a lesion area.

The stent delivery system of this application maintains accuracy of indwelling a stent and possesses high passing properties (i.e., ability to maneuver within the living body). This application also relates to a method of manufacturing the stent delivery system.

One aspect of the disclosure involves a stent delivery system including an elongated shaft, a balloon that is provided on an outer circumferential surface of a distal portion of the elongated shaft and is dilatable in a case where a fluid flows therein, and a stent that has a gap formed by wires, is formed to have a tubular shape in its entirety, is mounted on an outer circumferential surface of the balloon in a state where the balloon is contracted before dilation, and is deformed so as to increase in diameter due to dilation of the balloon. The balloon has a protruding portion which protrudes radially outward through the gap of the stent in a state where the stent is mounted. A lubricant is disposed in at least a part of the protruding portion. At least a part of a site covered with the stent of the balloon is provided with a site in which the lubricant is not disposed. Another aspect of the disclosure involves a stent delivery system that includes an elongated shaft, a balloon provided on the outer surface of the distal portion of the elongated shaft, and a stent formed by wires. The wires are spaced apart so that at least one gap is formed between the wires. The stent is tubular. The stent is mounted on the outer surface of the balloon when the balloon is in the contracted position before dilation. The stent is deformable to increase in outer diameter due to dilation of the balloon when the balloon is dilated to expand to the expanded position. The balloon includes a protruding portion which protrudes radially outward through the gap of the stent when the stent is mounted on the balloon and the balloon is in the contracted position. A lubricant is disposed on the outer surface of the balloon at least on a part of the protruding portion of the balloon. At least the outer surface of an other portion of the balloon that is covered by the stent is devoid of the lubricant

In the stent delivery system configured as described above, the stent is unlikely to deviate (separate) from the balloon since the lubricant is not disposed in at least a part of the outer surface of the balloon covered with the stent. Therefore, the accuracy of indwelling the stent can be maintained. Moreover, high passing properties (i.e., improved maneuverability within the living body) can be obtained because the protruding portion in which the lubricant is disposed comes into contact with a body lumen wall.

When a plurality of the protruding portions are provided along a circumferential direction of the balloon, the lubricant disposed in the protruding portion comes into contact with the body lumen wall across a wide range in the circumferential direction. Accordingly, the passing properties can be further enhanced, and the stent having significant sliding friction can be prevented from coming into contact with the body lumen wall as much as possible. Therefore, sliding friction of the stent against the body lumen wall can be restrained.

When the lubricants are disposed on the outer circumferential surfaces of the balloon and the elongated shaft respectively on a distal side and a proximal side relative to the stent, effects of high lubricity from the lubricant disposed in the protruding portion and high lubricity from the lubricants on the distal side and the proximal side of the stent are synergistically conducted. Therefore, higher passing properties (i.e., improved maneuverability within the living body) can be obtained.

When the protruding portion extends in a direction along the axial center of the elongated shaft, the lubricant disposed in the protruding portion comes into contact with the body lumen wall across a wide range in the axial center direction. Therefore, the passing properties (i.e., improved maneuverability within the living body) can be further enhanced.

When the stent is formed of a biodegradable material such as a biodegradable polymeric material and a biodegradable metal material, it is difficult to improve the passing properties because reducing the outer diameter of the stent to mount the stent on the balloon is difficult. However, even in such a stent made of a biodegradable material, high passing properties (i.e., improved maneuverability within the living body) can be obtained due to the lubricant disposed in the protruding portion.

Another aspect of the disclosure involves a method of manufacturing a stent delivery system in which a stent having a gap formed by wires and being formed to have a tubular shape in its entirety is disposed on an outer circumferential surface of a balloon being provided in a distal portion of an elongated shaft and being in a contracted state. The method of manufacturing a stent delivery system includes a mount step of mounting the stent on an outer surface of the balloon while the outer circumferential surface of the balloon is covered with the stent, the stent decreases in diameter, and the balloon protrudes radially outward through the gap of the stent; and a disposition step of disposing a lubricant in a protruding portion of the balloon protruding through the gap of the stent. The method of manufacturing the stent delivery system also may involve positioning a stent to axially overlap with and cover an outer surface of a balloon. The stent includes wires that are spaced apart from one another to define at least one gap between the wires. The balloon is connected to an elongated shaft. The method further includes mounting the stent on the outer surface of the balloon while the outer surface of the balloon is covered with the stent by decreasing the inner diameter of the stent, protruding a portion of the balloon radially outward through the at least one gap of the stent, and applying a lubricant on the outer surface of the protruding portion of the balloon that protrudes through the at least one gap of the stent.

In the method of manufacturing a stent delivery system described above, the protruding portion is formed when the stent is mounted on the balloon, and the lubricant is disposed in the protruding portion. Therefore, the lubricant is unlikely to be disposed in a site (portion) of the balloon covered with the stent, and the lubricant can be selectively and efficiently disposed in the protruding portion.

When the balloon partially dilates during mounting to form the protruding portion by injecting a fluid into the balloon while the stent decreases in diameter, the balloon can effectively protrude through the gap of the stent.

A further aspect of the disclosure involves a method of deploying a stent at a target site within a living body that includes inserting a distal end of an elongated shaft into a lumen in the living body. The distal end of the elongated shaft includes a balloon which possesses an outer surface. The stent is mounted on the outer surface of the balloon so that the stent surrounds an axial portion of the balloon. The outer surface of the balloon includes coated portions possessing a lubricant and non-coated portions not possessing the lubricant. The coated portions of the balloon and the non-coated portions of the balloon overlap one another in the axial direction. The method includes moving the distal end of the elongated shaft within the lumen in the living body. The coated portions of the balloon contacting the inner surface of the lumen in the living body as the distal end of the elongated shaft is moved within the lumen in the living body while at the same time the non-coated portions do not contact the inner surface of the lumen. The moving of the distal end of the elongated shaft within the lumen in the living body includes moving the distal end of the elongated shaft to position the stent at the target site in the living body. The method also includes inflating the balloon to expand the stent radially outward to deploy the stent at the target site within the living body.

### BRIEF DESCRIPTION OF THE DRAWINGS

FIG. 1 is a plan view of an embodiment of a stent delivery system.

FIG. 2 is an enlarged plan view of a balloon and a stent.

FIG. 3 is a cross-sectional view of the stent delivery system taken along line A-A in FIG. 2.

FIG. 4 is a longitudinal sectional view of a distal portion of the stent delivery system before the balloon of the stent delivery system dilates.

FIG. 5 is a longitudinal sectional view of the distal portion after the balloon of the stent delivery system dilates.

FIG. 6 is a schematic perspective view illustrating an apparatus manufacturing a stent delivery system.

FIG. 7 is a longitudinal sectional view that illustrates a state before the stent is mounted on the balloon by the apparatus manufacturing a stent delivery system.

FIG. 8 is a longitudinal sectional view that illustrates a state when the stent is mounted on the balloon by the apparatus manufacturing a stent delivery system.

FIG. 9 is a cross-sectional view that illustrates a state when the stent delivery system moves forward inside a blood vessel.

FIG. 10 is a longitudinal sectional view illustrating a modification example of the stent delivery system.

### DETAILED DESCRIPTION

Set forth below with reference to the accompanying drawings is a detailed description of embodiments of a stent delivery system, a method of use and manufacturing method representing examples of the inventive stent delivery system, method of use and manufacturing method disclosed here. Note that, for the convenience of description, there are cases where the dimensional ratios of the drawings are exaggerated and are different from the actual ratios.

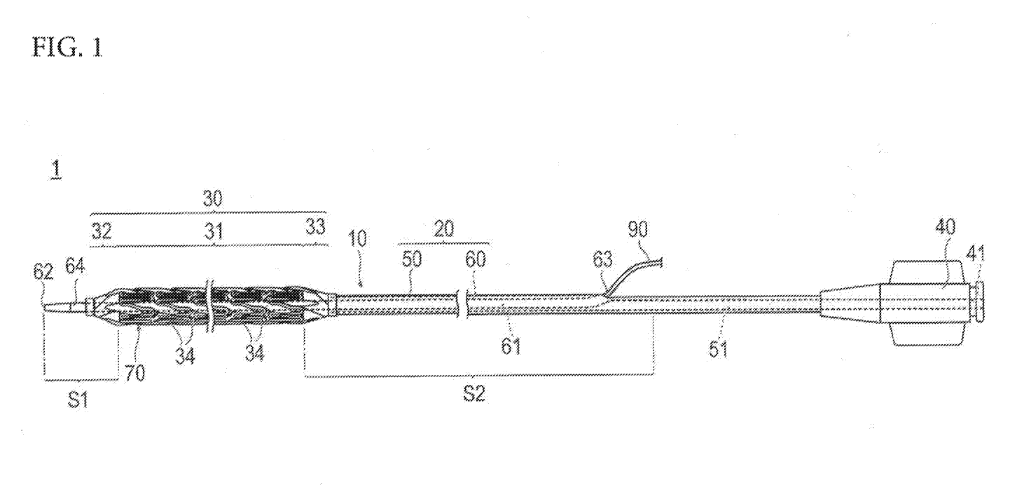

As illustrated in FIGS. 1 to 4, a stent delivery system 1 according to the present embodiment includes a balloon catheter 10 and a stent 70. The balloon catheter 10 is a device used for indwelling the stent 70 in a stenosed site (or an occluded site) that has occurred inside a blood vessel, the bile duct, the trachea, the esophagus, the urethra, and other body lumens. Note that, in this specification, a side to be inserted into a body lumen will be referred to as “distal end” or “distal side”, and the operating side for an operation by a user will be referred to as “proximal end” or “proximal side”.

The balloon catheter 10 has an elongated shaft 20 (i.e., shaft portion), a balloon 30 and a hub 40 which is fixed and attached to the proximal end of the elongated shaft 20. The balloon 30 is provided at a distal portion of the elongated shaft 20 and holds the stent 70.

The elongated shaft 20 includes an outer tube 50 that is a tubular body (i.e., cylindrical), and an inner tube 60 that is a tubular body disposed inside the outer tube 50. A dilation lumen 51 in which a dilation fluid circulates is formed inside the outer tube 50. The dilation fluid is for dilating the balloon 30 (i.e., expanding the balloon 30 radially outward). A guide wire lumen 61 is formed in the inner tube 60, and a guide wire is insertable through the guide wire lumen 61. The dilation fluid may be gas or may be liquid. Examples of dilation fluid gases include helium gas, CO₂ gas, and O₂ gas, and examples of dilation fluid liquids include saline (a physiological salt solution) and a contrast agent.

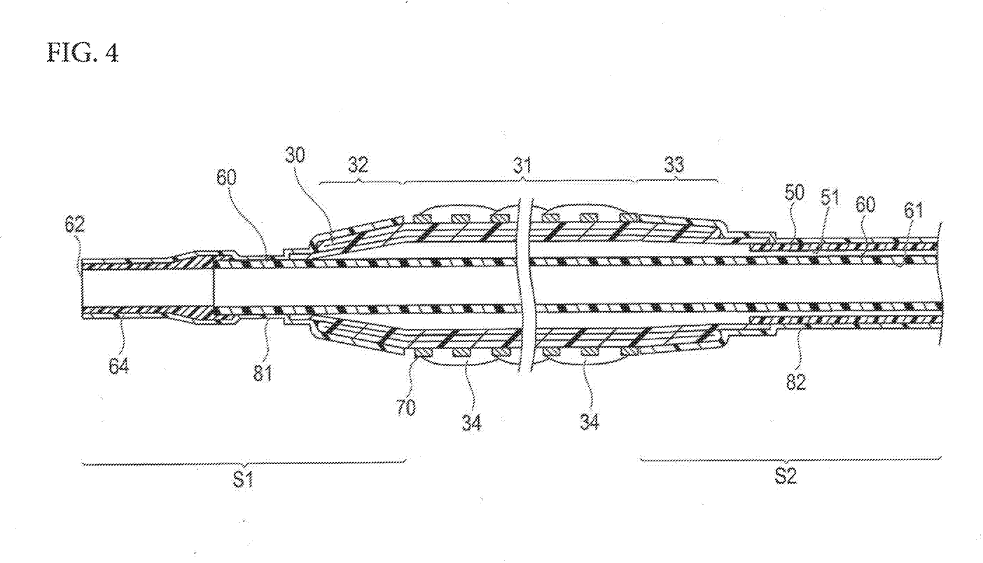

To reduce a burden to a living body that arises due to contact between the balloon catheter and an inner surface of a body lumen, a soft distal tip 64 is connected to the distal portion of the inner tube 60. As illustrated in FIG. 4, the inner tube 60 penetrates the inside of the balloon 30 (i.e., passes through the interior of the balloon 30) and is open at a distal opening portion 62 on the distal side of the balloon 30. A proximal portion of the inner tube 60 passes through the inner wall of the outer tube 50, is open at a proximal opening portion 63, and is fixed and attached to the outer tube 50 in a liquid-tight manner by using an adhesive or performing heat-welding. A lumen from the distal opening portion 62 to the proximal opening portion 63 forms the guide wire lumen 61.

The hub 40 includes a hub opening portion 41 which communicates with the dilation lumen 51 of the outer tube 50 and functions as a port through which the dilation fluid flows in and out. The proximal portion of the outer tube 50 is fixed and attached to the hub 40 in a liquid-tight manner by using an adhesive, performing heat-welding, or using a fastener (not illustrated).

It is preferable that the outer tube 50, the inner tube 60, and the distal tip 64 are formed of materials having a certain degree of flexibility. Examples of such a material include polyolefin such as polyethylene, polypropylene, polybutene, an ethylene-propylene copolymer, an ethylene-vinyl acetate copolymer, an ionomer, and a mixture of two or more kinds thereof; thermoplastic resin such as soft polyvinyl chloride resin, polyamide, a polyamide elastomer, polyester, a polyester elastomer, polyurethane, and fluororesin; silicone rubber; and latex rubber.

Examples of the hub 40 material include thermoplastic resin such as polycarbonate, polyamide, polysulfone, polyarylate, and a methacrylate-butylene-styrene copolymer.

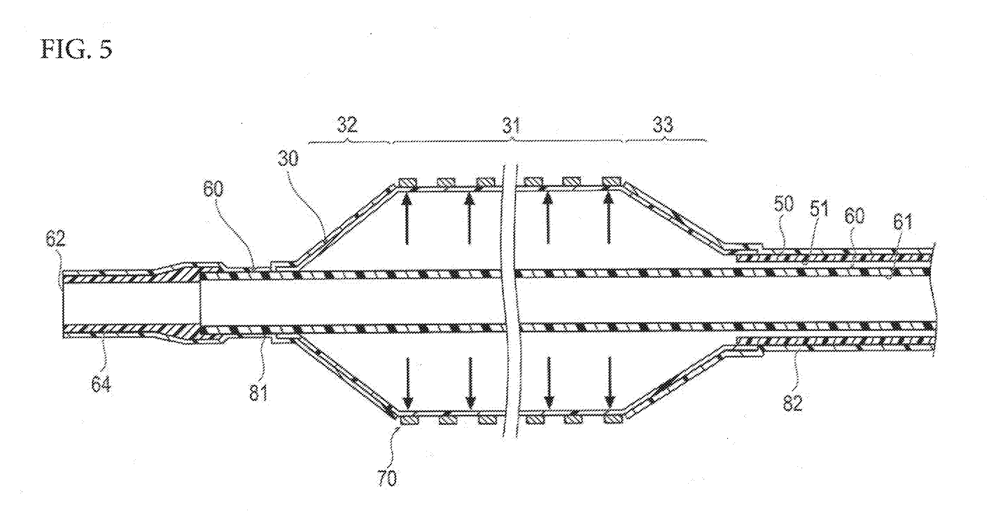

The balloon 30 widens the stent 70 when the balloon 30 dilates/inflates. In order to be able to efficiently widen a predetermined range, the balloon 30 has a tubular portion 31 which is formed at a central portion in an axial direction so as to have a substantially cylindrical shape and possess approximately the same diameter along the tubular portion 31 when the balloon 30 dilates. FIG. 5 illustrates an example of a tubular portion 31 possessing approximately the same outer diameter when the balloon 30 is inflated. The tubular portion 31 does not necessarily have a circular cross section orthogonal to the longitudinal axis. On the distal side of the tubular portion 31 of the balloon 30, a tapered distal portion 32 is formed, which decreases in outer diameter toward the distal side in a tapered manner (i.e., the outer diameter of the tapered distal portion 32 gradually decreases towards the distal end along the axial direction). On the proximal side of the tubular portion 31 of the balloon, a tapered proximal portion 33 is formed, which decreases in outer diameter toward the proximal side in a tapered manner (i.e., the outer diameter of the tapered proximal portion 32 gradually decreases towards the proximal end along the axial direction).

The distal side of the tapered distal portion 32 is fixed and attached to an outer wall surface of the inner tube 60 in a liquid-tight manner by using an adhesive, performing heat-welding, or the like. The proximal side of the tapered proximal portion 33 is fixed and attached to an outer wall surface of the distal portion of the outer tube 50 in a liquid-tight manner by using an adhesive, performing heat-welding, or the like. The inside of the balloon 30 thus communicates with the dilation lumen 51 formed in the outer tube 50, so that the dilation fluid can flow into the interior of the balloon 30 from the proximal side via the dilation lumen 51. The balloon 30 dilates (expands radially outwardly) when the dilation fluid flows into the interior of the balloon 30, and the balloon 30 folds and contracts (deflates radially inwardly) when the dilation fluid is discharged from the interior of the balloon 30.

As illustrated in FIGS. 1 to 4, the balloon 30 includes protruding portions 34 protruding radially outward through gaps formed between wires configuring the stent 70 (i.e., the outer surface of the protruding portions protrudes radially outwardly beyond the outer surface of the stent 70). Before dilation, the balloon 30 is in a folded state of being wound around the outer surface of the inner tube 60 in a circumferential direction. Such a balloon 30 can be formed inside a die by heating a tube (base material) and performing blow forming in which the tube is pressurized to expand from the inside by a fluid and is pressed to the die. The balloon may also be formed so as to be elastically deformed and have a widened outer diameter instead of being formed so as to be folded.

It is preferable that the balloon 30 material has a certain degree of flexibility. Examples of such a material include polyolefin such as polyethylene, polypropylene, polybutene, an ethylene-propylene copolymer, an ethylene-vinyl acetate copolymer, an ionomer, and a mixture of two or more kinds thereof; thermoplastic resin such as soft polyvinyl chloride resin, polyamide, a polyamide elastomer, polyester, a polyester elastomer, polyurethane, and fluororesin; silicone rubber; and latex rubber.

The stent 70 is a so-called balloon expandable stent which is plastically deformed and expands due to the dilating force applied by the balloon 30 when the stent 70 is installed (mounted) on the tubular portion 31 of the balloon 30. The stent 70 material may preferably be metal or resin having living-body suitability (i.e., a metal or resin that is suitable to be inserted into a living body). Examples of metal that are suitable to be inserted into a living body include an iron-based alloy such as stainless steel, tantalum (tantalum alloy), platinum (platinum alloy), gold (gold alloy), a cobalt-based alloy such as a cobalt-chromium alloy, a titanium alloy, and a niobium alloy. It is preferable to adopt a biodegradable metal material as the metal to be inserted into a living body, for example, magnesium. As the resin having living-body suitability, it is preferable to adopt a biodegradable polymeric material, that is, it is preferable to use a biodegradable synthetic polymer material such as polylactic acid, polyglycolic acid, a lactic acid-glycolic acid copolymer, polycaprolactone, a lactic acid-caprolactone copolymer, and poly-γ-glutamic acid; or a biodegradable natural polymer material such as cellulose and collagen.

The stent 70 is preferably a drug eluting stent. The outer surface of the stent 70 is preferably provided with a drug layer which includes a drug and elutes the drug inside a living body. Note that, the stent does not have to be the drug eluting stent.

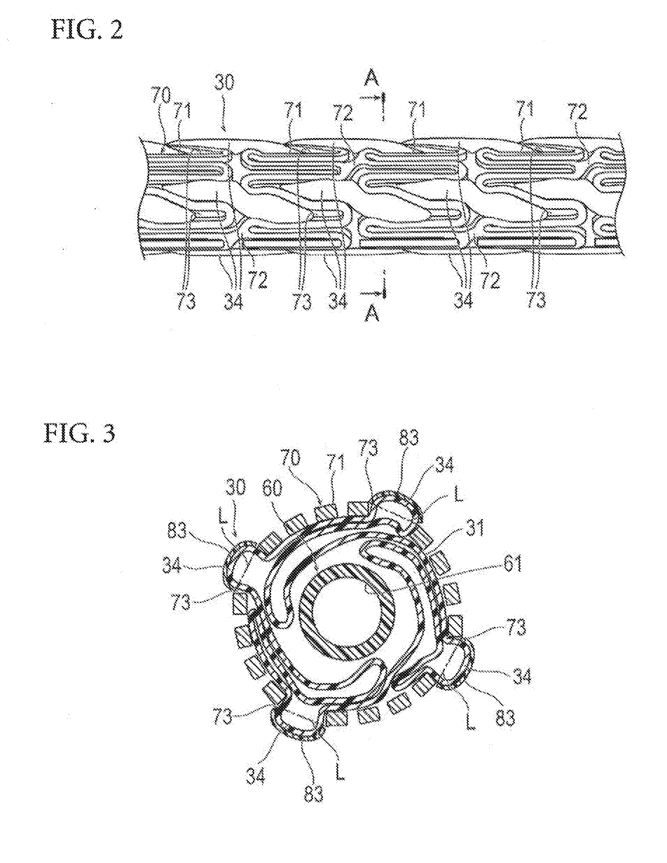

The stent 70 includes a plurality of annular portions 71 arranged in the axial direction. Each of the annular portions 71 is turned back (i.e., linear portions are bent/turned back as shown in FIG. 2) and formed so as to have an annular shape. The annular portions 71 are formed to possess gaps between the wires. The annular portions 71 have a tubular shape in its entirety when the annular portions 71 adjacent to each other are connected to each other by a link portion 72 (i.e., FIG. 2 illustrates that a plurality of annular portions 71 connected by link portions 72 possess a tubular shape along the entirety of the annular portions). When the stent 70 is contracted and being mounted on the balloon 30, large gap portions 73 wider than other gaps are formed in the stent 70. As illustrated in FIG. 5, the outer diameter of the stent 70 can increase when the balloon 30 dilates and each of the annular portions 71 is deformed such that the angle of the turned-back site increases (i.e., the gap between the linear portions of the annular portions 71 at the turned-back site is widened).

FIG. 3 illustrates that, for example, four large gap portions 73 are formed in the cross section orthogonal to the axis. The protruding portions 34 (i.e., a part of the balloon 30) protrude radially outward through the large gap portions 73. Since the large gap portions 73 are elongated in the axial direction, the protruding portions 34 are also formed to extend in the axial direction. In the cross section orthogonal to the longitudinal axis of the balloon 30, the protruding portions 34 protrude radially outward beyond a line L connecting the wires of the stent 70 interposing the large gap portions 73.

A plurality of the protruding portions 34 of the balloon 30 and a plurality of the large gap portions 73 of the stent 70 are preferably provided along the circumferential direction of the balloon 30. In the embodiment illustrated in FIG. 3, four of the protruding portions 34 are provided at substantially equal angles (90°) in the circumferential direction. However, the number of protruding portions 34 is not limited. It is also preferable to provide one or more protruding portions 34 within a range of 180° in the circumferential direction. However, the angle range is not limited. The protruding portions 34 are preferably disposed at equal angles in the circumferential direction. However, the angles between respective protruding portions 34 do not have to be equal to each other. Each of the protruding portions 34 is formed to extend in a direction along an axial center of the elongated shaft 20 in a relatively long and relatively thin manner. However, the shape of the protruding portions 34 is not limited.

As illustrated in FIGS. 1 and 4, the outer surface of a range S1 including the tapered distal portion 32 of the balloon 30, the inner tube 60 on the distal side of the tapered distal portion 32, and the distal tip 64 is coated with a first lubricating layer 81 formed of a lubricant. In addition, the outer surface of a range S2 including the tapered proximal portion 33 of the balloon 30 and a predetermined length of the elongated shaft 20 in a proximal direction from the tapered proximal portion 33 is coated with a second lubricating layer 82 formed of a lubricant. The proximal opening portion 63 of the guide wire lumen 61 is preferably positioned in the range S2.

The outer surfaces of the protruding portions 34 of the balloon 30 are coated with a third lubricating layer 83 formed of a lubricant (i.e., so that the outer surface of the balloon includes coated portions possessing a lubricant and non-coated portions not possessing the lubricant, the coated portions of the balloon and the non-coated portions of the balloon overlapping in the axial direction as shown in FIG. 2). Note that, a portion of the tubular portion 31 of the balloon 30 covered with the stent 70 (that is, a site between the balloon 30 and the wires configuring the stent 70) is preferably not coated with a lubricant. However, the portion of the balloon 30 may be partially coated with a lubricant. The outer surface of the stent 70 is preferably not coated with a lubricant. When the stent 70 expands and is indwelled in a vascular wall while the stent 70 is not coated with a lubricant, the stent 70 is unlikely to slide with respect to the vascular wall, thereby being able to be accurately indwelled. In addition, when the stent 70 is a drug eluting stent 70, a drug is more likely to act on a vascular wall because the stent 70 is not covered with a lubricant.

The lubricant may be, for example, a hydrogel mixture of polyethylene glycol and a hydrophilic polymer material. Polymers used as the lubricant are non-cross-linking water-soluble polymers having a chain structure containing a hydrophilic group such as —OH, —CONH2, —COOH, —NH2, —COO—, —SO3, and —NR3+ (here, R is alkyl or hydrogen).

Natural water-soluble polymers such as carboxymethyl cellulose (CMC), methylcellulose (MC), hydroxyethyl cellulose (HEC), and hydroxypropylcellulose (HPC) can also be used for the lubricant. In addition, in the lubricant, synthetic water-soluble polymers such as polyethylene oxide, polyethylene glycol, and methoxy polyethylene glycol can also be used together with maleic anhydride polymers, for example, methyl vinyl ether-maleic anhydride copolymers. Moreover, water-soluble nylon and pyrrolidones, for example, polyvinylpyrrolidone can also be used as the lubricant. A derivative of the polymers is not limited to a water-soluble derivative. However, the derivative has the above-described water-soluble polymers as a basic configuration. The water-insoluble derivative has free chain molecules and can be used as long as the derivative is hydrophilic.

It is further possible to use, as the lubricant, esterified polymers, salts, amides, anhydrides, halides, ethers, hydrolysates, acetals, formals, alkyloles, quaternary polymers, diazos, hydrazides, sulfonic acids, nitrates, and ionic acid compounds which are obtained through condensation, addition, substitution, oxidation, or reduction reaction of the above-described water-soluble polymers. It is possible to use polymers cross-linking with a substance having one or more reactive functional groups of a diazonium group, an azide group, an isocyanate group, an acid chloride group, an acid anhydride group, an iminocarbonate group, an amino group, a carboxyl group, an epoxy group, a hydroxyl group, and an aldehyde group. It is also possible to use copolymers having vinyl compounds, acrylic acid, methacrylic acid, diene compounds, and maleic anhydride as the lubricant.

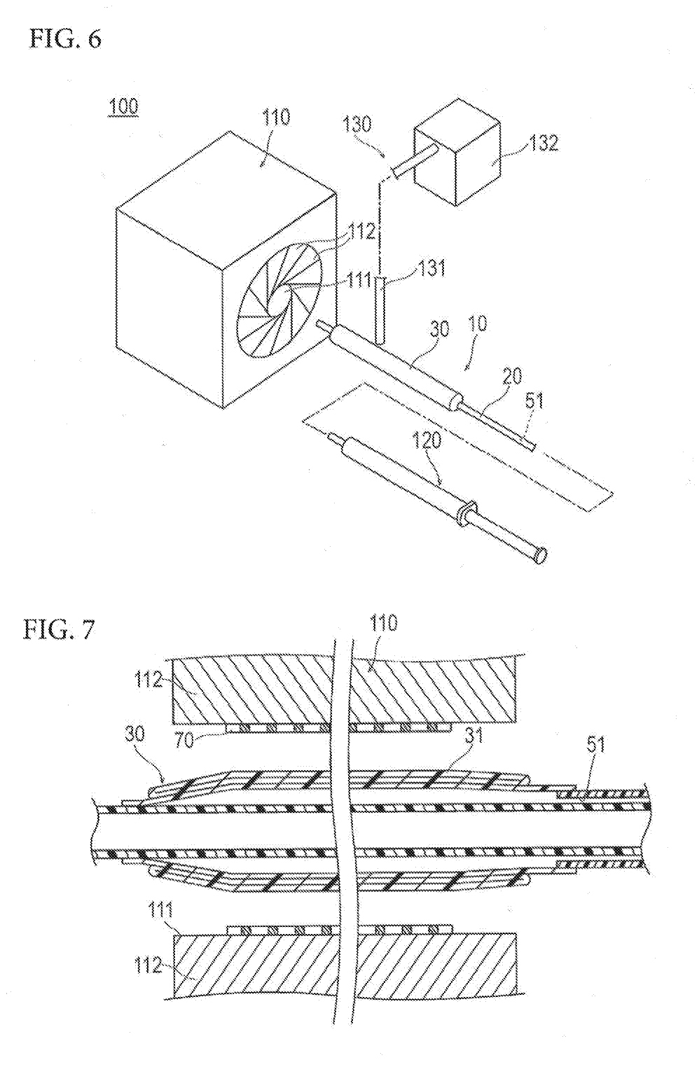

Next, a manufacturing apparatus 100 for manufacturing the stent delivery system 1 will be described. As illustrated in FIG. 6, the manufacturing apparatus 100 includes a fixing portion 110 for mounting the stent 70 on the balloon 30, a fluid supply section 120 which supplies a fluid into the balloon 30 via the dilation lumen 51, and a lubricant supply section 130 which causes the lubricant to be attached to the balloon 30.

A general crimping tool can be used as the fixing portion 110 for crimping the stent 70 on the balloon 30 of the balloon catheter 10. The fixing portion 110 can include a plurality of movable members 112 arranged in the circumferential direction to form a chamber 111 which can decrease in inner diameter as shown in FIG. 6. The inner diameter of the chamber 111 can increase and decrease by relatively moving the plurality of movable members 112.

The fluid supply section 120, for example, is a syringe, an indeflator, or a pump. The fluid supply section 120 is interlocked with the hub opening portion 41 and supplies a fluid into the interior of the balloon 30 via the dilation lumen 51.

The lubricant supply section 130 includes a nozzle 131 configured to discharge a solution of a lubricant which has dissolved in a solvent. The lubricant supply section 130 also includes a pump 132 configured to supply the solution to the nozzle 131. Examples of the solvent include alcohols, aliphatic hydrocarbons, aromatic hydrocarbons, a chlorinated solvent, esters, glycols, glycol ethers, and ketones. Examples of a polar solvent include alcohols, glycols, and water. More specific examples include ethanol, methanol, isopropanol, stearyl alcohol, ethylene glycol, propylene glycol, glycerin, and water. Examples of a nonpolar solvent include aliphatic hydrocarbons such as heptane and hexane; aromatic hydrocarbons such as toluene and xylene; chlorinated hydrocarbons such as perchloroethylene, methylene chloride, chloroform, carbon tetrachloride, and 1,1,1-trichloroethane; fluorocarbons; and mineral spirits.

Next, a method of manufacturing the stent delivery system 1 according to the present embodiment will be described.

First, the stent 70 is inserted into the chamber 111 while the inner diameter of the chamber 111 of the fixing portion 110 is greater than the outer diameter of the stent 70. Thereafter, the movable members 112 are moved to decrease the inner diameter of the chamber 111 so that the stent 70 is held by the movable members 112. Next, as illustrated in FIG. 7, the balloon 30 is positioned inside the stent 70 while the stent 70 is held by the movable members 112.

The movable members 112 are further moved to decrease the inner diameter of the chamber 111. The inner surface of the stent 70 is thus brought into contact with the outer circumferential surface of the balloon 30 by decreasing the inner diameter of the stent 70. The fluid supply section 120 (refer to FIG. 6) is then used to supply fluid, as illustrated in FIG. 8, into the balloon 30 via the dilation lumen 51. Accordingly, the internal pressure inside the balloon 30 rises, and the balloon 30 enters (protrudes radially outwardly into) the large gap portions 73 of the stent 70. Thereafter, the movable member 112 is moved to further decrease the inner diameter of the chamber 111.

Fluid is then discharged from the inside of the balloon 30 by using the fluid supply section 120. The movable members 112 are moved so as to dilate (expand) the inner diameter of the chamber 111. Accordingly, the stent 70 maintains a state of being mounted on the outer surface of the contracted balloon 30 (mount step). At this time, the protruding portions 34 of the balloon 30 protrude radially outward through the large gap portions 73 of the stent 70.

Next, the balloon catheter 10 and the stent 70 are taken out from the fixing portion 110. The nozzle 131 is moved/positioned to approach the protruding portions 34. A solution containing a lubricant is then supplied from the nozzle 131 to be applied (adheres) to the protruding portions 34. The solution dries to form the third lubricating layer 83 disposed on the outer surface of the protruding portions 34 (disposition step). The nozzle 131 is next moved/positioned at the distal tip 64, the inner tube 60, the tapered distal portion 32, the tapered proximal portion 33, and the outer tube 50. The solution containing the lubricant is supplied from the nozzle 131 and is dried, thereby forming the first lubricating layer 81 and the second lubricating layer 82. Accordingly, the stent delivery system 1 according to the present embodiment is manufactured.

The method of disposing a lubricant is not limited to the above-described method. For example, masking processing can be performed at a site in which a lubricant is not intended to be disposed, and after the balloon 30 in its entirety is immersed in the solution containing the lubricant, the balloon 30 is picked up, thereby drying the adhered solution. Accordingly, it is possible to form a lubricating layer in only a site which is not subjected to masking.

In the above-described method of manufacturing the stent delivery system 1, the protruding portions 34 are formed when fixing the stent 70 to the balloon 30, and lubricants are disposed on the outer surface of the protruding portions 34. Therefore, the lubricant is unlikely to be disposed in a part of the balloon 30 that is covered with the stent 70. The lubricant can be selectively and efficiently applied to the protruding portions 34.

While the stent 70 decreases in diameter due to crimping, a fluid is injected into the balloon 30 and the protruding portions 34 are formed by partially dilating the balloon 30. Therefore, the balloon 30 can efficiently protrude through the gaps of the stent 70.

Next, an operation of the stent delivery system 1 is described based on an example case of being used in treatment of a stenosed site in a blood vessel in a living body.

Before performing treatment of a stenosed site in a blood vessel, air inside the balloon 30 and the dilation lumen 51 is deflated (released) so that the air inside the balloon 30 and the dilation lumen 51 is replaced by the dilation fluid. At this time, the balloon 30 is in a folded state, the stent 70 is installed (mounted) on the outer circumference of the balloon 30, and the protruding portions 34 of the balloon 30 protrude through the large gap portions 73 of the stent 70 (refer to FIGS. 1 to 4).

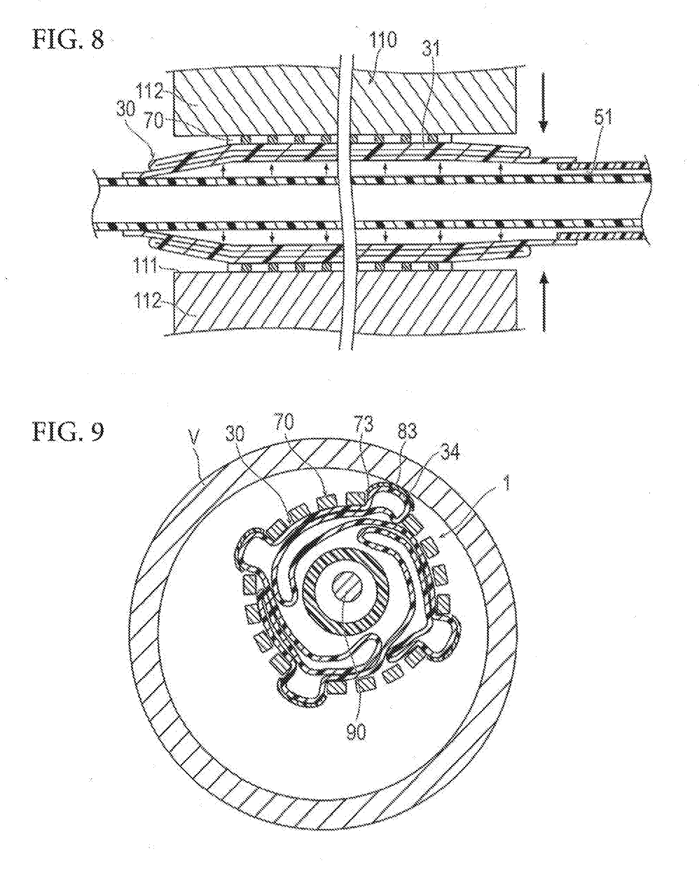

Next, for example, a sheath is indwelled in a blood vessel V of a patient by the Seldinger method. When a guide wire 90 is inserted through the inside of the guide wire lumen 61, the guide wire 90 and the stent delivery system 1 are inserted into the blood vessel V through the inside of the sheath (not illustrated). Subsequently, as illustrated in FIG. 9, while the guide wire 90 takes the lead inside the blood vessel V, the stent delivery system 1 moves forward, and the balloon 30 arrives at a stenosed site. As discussed above, the third lubricating layer 83 is disposed on the outer surface of the protruding portions 34 of the balloon 30 protruding radially outward through the large gap portions 73 of the stent 70. The passing properties (i.e., relative ease of moving within the living body) of the stent delivery system 1 can be improved due to the third lubricating layer 83 coming into contact with a vascular wall (body lumen wall) even at a place through that is more difficult for the stent delivery system to pass, such as a bent portion, a bifurcated portion, or a stenosed site of a blood vessel.

Since a plurality of protruding portions 34 are provided along the circumferential direction of the balloon 30, the third lubricating layer 83 on the outer surface of the protruding portions 34 comes into contact with a vascular wall across a wide range in the circumferential direction. Accordingly, the passing properties can be further enhanced. A stent 70 that will create significant sliding friction with a vascular wall can thus be prevented from coming into contact with the vascular wall as much as possible. Therefore, sliding friction generated between the stent and a vascular wall can be restrained.

Since the first lubricating layer 81 and the second lubricating layer 82 (refer to FIG. 4) are formed on the outer circumferential surfaces of the balloon 30 and the elongated shaft 20 respectively on the distal side and the proximal side relative to the stent 70, an effect of high lubricity from the third lubricating layer 83 of the protruding portions 34 and effects of high lubricity of the first lubricating layer 81 and the second lubricating layer 82 are synergistically conducted. In other words, the first and third lubricating layers 81, 83 further improve the maneuverability of the stent delivery system by decreasing the friction that may arise when the outer surface of a portion of the stent delivery system 1 contacts a vascular wall. Therefore, higher passing properties can be obtained.

The third lubricating layer 83 on the outer surface of the protruding portions 34 comes into contact with a vascular wall across a wide range in the axial direction because the protruding portions 34 are formed to extend axially along the elongated shaft 20. Therefore, the passing properties of the stent delivery system 1 can be further enhanced.

When the stent 70 is formed of a biodegradable material (such as a biodegradable polymeric material and a biodegradable metal material), it may be more difficult to improve the passing properties since the outer diameter of the stent 70 is unlikely to be small in a case of mounting the stent 70 on the balloon 30. However, high passing properties can be obtained by the third lubricating layer 83 disposed on the protruding portions 34.

After the balloon 30 is disposed in a stenosed site, a predetermined amount of the dilation fluid is injected through the hub opening portion 41 of the hub 40 by using an indeflator, a syringe, or a pump, and the dilation fluid is sent into the balloon 30 through the dilation lumen 51. Accordingly, the folded balloon 30 dilates as illustrated in FIG. 5. The tubular portion 31 of the balloon 30 thus expands to cause the stent 70 to be plastically deformed radially outwardly to widen the stenosed site.

Thereafter, the dilation fluid is aspirated and discharged through the hub opening portion 41, and the balloon 30 is deflated to be in a folded state. Accordingly, the stent 70 which is plastically deformed and expanded radially outwardly is indwelled in the stenosed site while being in a dilated state. Thereafter, the guide wire 90 and the stent delivery system 1 are removed from the blood vessel via the sheath, and the stent delivery is complete.

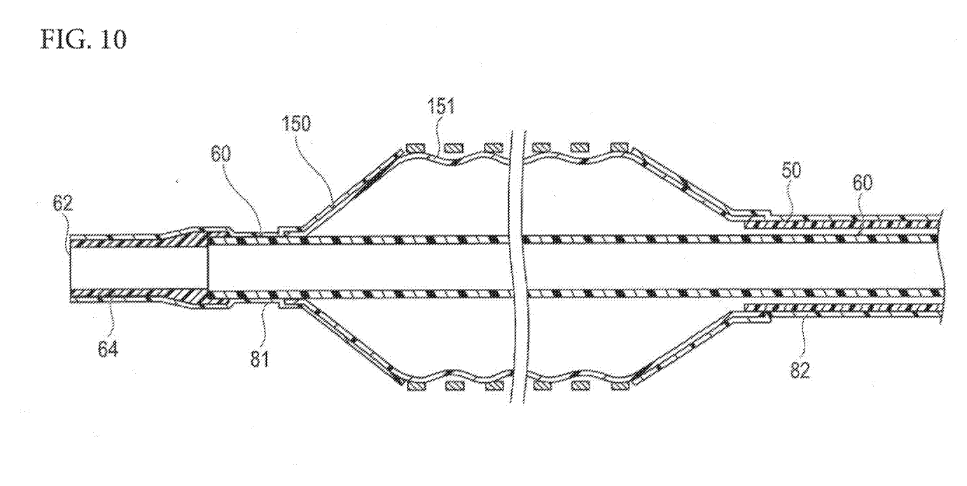

The disclosed stent delivery system, stent delivery system manufacturing method, and method of deploying the stent is not limited to only the embodiments described above. Various changes can be made by those skilled in the art within the technical idea of the present disclosure. For example, the outer diameter of the tubular portion of the balloon does not have to be uniform. For example, a tubular portion 151 of a balloon 150 may be formed so as to have an irregular shape in which the outer diameter varies in a bellows shape as in a modification example illustrated in FIG. 10.

The structure of the stent is not particularly limited as long as there is an aperture through which a part of the balloon can protrude when the stent is mounted on the balloon.

The detailed description above describes a stent delivery system and a method of manufacturing a stent delivery system. The invention is not limited, however, to the precise embodiments and variations described. Various changes, modifications and equivalents can be effected by one skilled in the art without departing from the spirit and scope of the invention as defined in the accompanying claims. It is expressly intended that all such changes, modifications and equivalents which fall within the scope of the claims are embraced by the claims.

What is claimed is:

1. A stent delivery system comprising:
an elongated shaft comprising a distal portion and possessing an outer surface;
a balloon provided on the outer surface of the distal portion of the elongated shaft, the balloon being dilatable from a contracted position to an expanded position when a fluid flows into an interior of the balloon, the balloon possessing an outer surface;
a stent formed by wires, the wires being spaced apart so that at least one gap is formed between the wires, the stent possessing a tubular shape;
the stent being mounted on the outer surface of the balloon when the balloon is in the contracted position before dilation, the stent being deformable to increase in outer diameter due to dilation of the balloon when the balloon is dilated to expand to the expanded position;
the balloon comprises a protruding portion which protrudes radially outward through the gap of the stent when the stent is mounted on the balloon and the balloon is in the contracted position; and
a lubricant disposed on the outer surface of the balloon at least on a part of the protruding portion of the balloon, and at least the outer surface of an other portion of the balloon that is covered by the stent being devoid of the lubricant.

2. The stent delivery system according to claim 1, wherein a plurality of the protruding portions are provided along a circumferential direction of the balloon, the protruding portions being spaced apart from one another in the circumferential direction.

3. The stent delivery system according to claim 2, wherein
the stent possesses an outer diameter when the stent is mounted on the outer surface of the balloon and the balloon is in the contracted position,
the balloon possesses an outer diameter at the protruding portions when the balloon is in the contracted position, and
the protruding portions protrude radially outward beyond the wires of the stent so that the outer diameter of the balloon at the protruding portions is greater than the outer diameter of the stent.

4. The stent delivery system according to claim 1, wherein the lubricant is disposed on the outer surface of the balloon distal to the stent and on the outer surface of the balloon proximal to the stent.

5. The stent delivery system claim 1, wherein the elongated shaft extends in an axial direction and the protruding portion extends in the axial direction.

6. The stent delivery system according claim 1, wherein the stent is formed of a biodegradable material.

7. A method of manufacturing a stent delivery system comprising:
positioning a stent to axially overlap with and cover an outer surface of a balloon, the stent comprising wires that are spaced apart from one another to define at least one gap between the wires, the balloon being connected to an elongated shaft;
mounting the stent on the outer surface of the balloon while the outer surface of the balloon is covered with the stent by decreasing the inner diameter of the stent;
protruding a portion of the balloon radially outward through the at least one gap of the stent; and
applying a lubricant on the outer surface of the protruding portion of the balloon that protrudes through the at least one gap of the stent.

8. The method of manufacturing the stent delivery system according to claim 7, wherein the balloon partially dilates during the protruding to form the protruding portion by injecting a fluid into the balloon while the stent has the decreased inner diameter.

9. The method of manufacturing the stent delivery system according to claim 7, wherein the protruding of the portion of the balloon comprises forming a plurality of circumferentially spaced apart protruding portions of the balloon.

10. The method of manufacturing the stent delivery system according to claim 9, wherein at least some of the plurality of circumferentially spaced apart protruding portions of the balloon are axially spaced apart from one another.

11. The method of manufacturing the stent delivery system according to claim 7, wherein the mounting of the stent is performed by crimping the stent onto the outer surface of the balloon.

12. The method of manufacturing the stent delivery system according to claim 7, further comprising:
applying the lubricant on the outer surface of the balloon distal to the stent and on the outer surface of the balloon proximal to the stent.

13. A method of deploying a stent at a target site within a living body comprising:
inserting a distal end of an elongated shaft into a lumen in the living body, the elongated shaft extending in an axial direction, the distal end of the elongated shaft comprising a balloon which possesses an outer surface, the stent being mounted on the outer surface of the balloon so that the stent surrounds an axial portion of the balloon, the outer surface of the balloon comprising coated portions possessing a lubricant and non-coated portions not possessing the lubricant, the coated portions of the balloon and the non-coated portions of the balloon overlapping one another in the axial direction;
moving the distal end of the elongated shaft within the lumen in the living body, the lumen possessing an inner surface, the coated portions of the balloon contacting the inner surface of the lumen in the living body as the distal end of the elongated shaft is moved within the lumen in the living body while at the same time the non-coated portions do not contact the inner surface of the lumen;
the moving of the distal end of the elongated shaft within the lumen in the living body comprising moving the distal end of the elongated shaft to position the stent at the target site in the living body; and
inflating the balloon to expand the stent radially outward to deploy the stent at the target site within the living body.

14. The method according to claim 13, further comprising:
deflating the balloon and removing the elongated stent and the balloon from within the living body, while the stent remains deployed at the target site within the living body.

15. The method according to claim 13, wherein the coated portions of the balloon protrude radially outwardly beyond the non-coated portions of the balloon.

16. The method according to claim 13, wherein at least some of the coated portions of the balloon are circumferentially spaced apart from one another.

17. The method according to claim 13, wherein the coated portions of the balloon contacting the inner surface of the lumen in the living body are positioned between opposite axial ends of the stent.

18. The method according to claim 13, wherein the coated portions of the balloon contacting the inner surface of the lumen in the living body include coated portions of the balloon that are axially spaced apart from one another.
